(19) United States
(12) Patent Application Publication
(10) Pub. No.: US 2021/0030641 A1
(43) Pub. Date: Feb. 4, 2021

(54) Intravenous Bolus Subdermal Facial

(71) Applicant: Vivi R. Stafford, Mission Viejo, CA (US)

(72) Inventor: Vivi R. Stafford, Mission Viejo, CA (US)

(21) Appl. No.: 16/902,242

(22) Filed: Jun. 15, 2020

(51) Int. Cl. A61K 8/19; A61Q 19/08; A61Q 7/00; A61Q 19/02

(52) U.S. Cl. CPC A61K 8/19; A61Q 19/02; A61Q 7/00; A61Q 19/08

(57) ABSTRACT

A method for injecting aqueous solutions as a bolus of cells below the dermis, into the hypodermis and the surrounding tissues for more profound cosmetic outcomes than a topical approach. The application of intravenous bolus subdermal solutions can not only nurture the skin but for cases where the skin is not nurtured or hydrated by this application the side effects of the aspects that cause local injury or irritation can also upregulate healthy skin cells and biological cells and tissues related to the skin to improve the health and appearance of the skin and its neighboring tissues and structures.

### BACKGROUND OF THE INVENTION

This invention was not made with U.S. government support.

Methods of regenerating human tissues often comes from.

A facial is essentially a multi-step skin treatment that is one of the best ways to take care of your skin. A facial cleanses, exfoliates, and nourishes the skin, promoting a clear, well-hydrated complexion and can help skin appear younger or be physiologically healthier. Methods of multi-step skin treatments subdermal or subcutaneously as an approach to giving a more aggressive facial is not currently documented anywhere in the capacity of giving a full facial including and not limited to hydration steps, peeling effects, dermabrasion effects or nutritional effects and to correct defects such as acne, hyperpigmentation, irritation, small lines and wrinkles. There are documents of subcutaneous treatments for skin pathology but zero for subcutaneous treatments that are volume dependent with aqueous solutions for the sole purpose of providing for facial results with the goal of more profound results than what are traditionally found with topical facial methods, the current standard. The subdermal approach of volume injected under the skin as a bolus or in the method of a facial provides for substrates or molecules that the dermis, hypodermis and other structures below the dermis can utilize for the generation of new tissues in an array of cellular pathways. After the onset of puberty, the dynamics of skin healing and rejuvenating skin properties and the proximate structures of the hypodermis experienced since birth begins to change early in life; meanwhile, the process of skin denaturing continues throughout life. Skin cells have life spans where old skin cells exfoliate and the newer generated cells do not incorporate the same amount of health as the previous skin cells may have. Over time, the quality of the skin via the skin itself and the structures below the dermis change. However, through a healthier dermis and hypodermis similarly found in youth can preserve the quality of the skin and proximate structures. This can be done through regeneration methods such as subdermal hydration in large boluses in the approach of providing a subdermal facial. Blood flow is considered essential for life and the micro vessels can become engorged and less effective. Processes of angiogenesis can preserve the health of the dermis and the health of the structures within the hypodermis; such as, hair follicles. Dermal papilla for instance, is known for its support of the hair follicle; however, there are no proven methods in maintaining the health of the dermal papilla.

Currently there are no approved or rigorously evaluated methods for causing medical grade facial injection boluses to the hypodermis or below the epidermis using injectable methods for the purposes of techniques encompassing a topical facial. Methods have been in place for approaching cell regeneration using laser technology or intense light technology or through methods related to thermoregulation outside of typical topical facial results. However, there are no methods to create regeneration of the skin or to have facial like results by keeping the epidermis fully intact and by approaching skin regeneration with aqueous bloused solutions injected into the hypodermis for therapeutic regenerative tissues as a facial or through facial techniques. There have been concerns over facials giving non-medical grade results whereas the subdermal injection of the fluids provides more direct contact of hydration fluids, peeling agents and low viscosity preparations for the purposes of skin healing with laser methods which generally are approached on top of the dermis. In relations to laser therapy or other topical methods of providing for facials, there has been complications from those methods such as blistering of the epidermis and post inflammatory hyperpigmentation which a subdermal or hypodermal approach is far less likely to provide. Approaches below the dermis are more likely to give profound results from a facial and far less likely to accomplish full thickness damage to the skin.

The demand for topical facial aqueous solutions placed on top of the dermis is very high. In regards to injections of facial aqueous solutions below the dermis as a skin corrective measure and for angiogenesis purposes including preserving the anagen phase in regards to follicles in the papillary dermis is very high in the cosmetic market and also in those with issue in relations to thinning hair or hair loss and those in need of rejuvenating facial results. Laser therapy is very expensive with risk of side effects, dermabrasions have a true risk for post inflammatory hyperpigmentation and other untoward events from the current methods in use. This method provides for gentle microdermabrasion like techniques through bolus injections below the dermis.

In spite of procedural risks which are low, the growing appeal of cosmetic medical treatments is a testament to the psychological importance of body image and the beneficial outcomes of the procedures as perceived by the populations who seek therapy for their pathology, skin irregularities or for the enhancement they would like to achieve through traditional facials which can be more rigorously achieved through a subdermal approach to a facial via bolus injections. The significance of a product that delivers outcomes through a subdermal bolus for the approach of a facial with little to no risk to the epidermis or superficial dermis is significant.

### SUMMARY OF THE INVENTION

The present invention is directed to a method for a non-surgical approach and a non-laser or heat modulated approach and a non-facial approach but a subdermal approach to cause more profound facial hydration through injections into the hypodermis. The injections would be with aqueous solution consistent with water at −2 to 42 Celsius. The solution would be provided within the context of a facial. The aqueous acid solutions which would comprise of water solutions for cleansing purposes, salt water solutions for hypodermal foliation purposes, are injected below the structures of the dermis and within the hypodermis and it neighboring tissues where either it will provide nourishing, hydrating, irritating or denaturing reversible and also irreversible cellular damage may occur causing hyperemia. In response to the irritation and injury the hypodermis will trigger a healing response which will sequester cells for regenerating the skin and angiogenesis will also occur. The advantage of this treatment is to cause facial skin rejuvenating results similar to a laser with techniques of injections of aqueous solutions that are hyperemia causing and clinically controlled corrosion in tissues below the dermis. This method also providing for nourishment to the tissues Previously clinical problems and side effects from facials that approach the skin from the epidermis can be challenging to the provider and the recipient where these subdermal approaches quell the risks to long term unwanted results. These methods are from using a hypodermic needle and applying facial solutions below the dermis with a needle or another device for providing bolus solutions below the dermis. Through this approach the skin heals deep and there is potentially very little down time with approach being the below the dermis. In addition, this approach causes leaky vessels and allows plasm leakage which means that droplets of autologous blood or exogenous blood can be used for similar results or better results in current platelet rich plasma treatments.

The foregoing, and other features and advantages of the invention, will be apparent from the following, more particular description of the embodiments of the invention, the accompanying drawings, and the claims.

I claim:

1. A method of Injecting with a hypodermic needle aqueous solutions for purposes of a deep facial in human skin below the epidermis and into the hypodermis of the skin specifically through caustic injury and gentle injury or nourishment to the hypodermis with facial solutions which include but are not limited to buffered acid solutions with the viscosity of aqueous solutions such as water and buffered alkali solutions, salt solutions, vitamin solutions for the purposes of subdermal facials. Injections intent to cause a subclinical burn or burns in the subdermal plain, hypodermis causing localized injury, denaturing tissue and causing hyperemia through the acid or alkali caustic solutions within the scope of a bolused styled facial sub dermally or through gentler nutritional, oxygenation or nutritious support.

2. The method of claim one where Injections of caustic agents or non-caustic agents for bolus injections into the hypodermis which causes capillary permeability due to the effects of the damage from the bolused substance and where Injections of caustic agents or facial styled agents causes alkali burns of the hypodermis which causes capillary permeability due to the effects of the damage from the alkalotic chemical.

3. The method of claim one where bolus Injections of the buffered solution of the acidic origin causes injury resulting in plasma leaks. This plasma leaks encourage angiogenesis and where buffered solution of the alkaline origin causes injury resulting in plasma leaks. This plasma leaks encourage angiogenesis. And where Injections of buffered acid solutions causes plasma permeability and also causes the aggregation of fibrin encouraging angiogenesis and where Injections of buffered alkali solutions causes plasma permeability and also causes the aggregation of fibrin encouraging angiogenesis.

4. The method of claim one requires injection methods below the dermis. The method of claim one where Injections of the buffered alkali solutions increases capillary leak ability and plasma permeability allowing for effective droplet treatments of exogenous or autologous plasma or packed cells or red blood and where Injections of the buffered alkali solutions increases capillary leak ability and plasma permeability allowing for effective droplet treatments of exogenous or autologous plasma or packed cells or red blood cells. The method of claim one where Injections of the buffered alkali and buffered acid solutions increases capillary leak ability and plasma permeability for effective treatments with droplets treatments of autologous or exogenous plasma or red blood cells or packed cells.

5. The method of claim one where the bolused facial goal is for wound healing purposes, cosmetic purposes including treating discoloration in the skin, hyperpigmentation, hypopigmentation, treating pores, smoothing textures, improving clarity and tone, treating acne and smoothing fine lines and will improve the overall skin health including brightening the skin and decreasing the appearance of cellulite.

6. The method of claim one where the facial bolus is for the purpose enhancing the condition of the subdermal tissues, hypodermal tissues including the subcutaneous tissue by increasing the permeability of plasma and the leak-ability of capillaries for up to 48 hours with subsequent activation of clotting cascade and the pooling of fibrin in clot formation causing the appearance of volumizing the hypodermis and subdermal tissues hence improving the appearance of deep folds, dimples, crevices, folds, wrinkles and enhancing the overall appearance of the three dimensionality of the skin.

7. The method of claim one where the facial fluid bolus is for the purposes of hair growth through the stimulation of angiogenesis and hyperemia as it relates to hydration from the bolus and the irritation caused by the subdermal and hypodermal fluid bolus is for the purposes of hair growth through the triggering of capillary permeability and the subdermal and hypodermal facial fluid bolus is for the purposes of hair growth through the enhancement of plasma leak ability, hyperemia and angiogenesis with the intravenous fluid bolus placed subdermally.

8. The method of claim one where inflammatory and reactive, proliferative and reparative and maturation and remodeling improves the overall condition and health of the skin without full thickness injury to the dermis through the facial fluid bolus. Including the method of claim one includes buffered acid solutions with the viscosity of water. And the method of claim one includes buffered caustic solutions with the viscosity of water with or without micro particles and is not an injectable implant for the purposes of a subdermal facial.

9. The method of claim one includes solutions and polymerized solutions of caustic agents or non-caustic agents in a bolus fashion and denatured solutions with the viscosity of water and solutions with the viscosity of waters with micro particles for the purposes of foliation for the scalp and the skin and the structures in nourishes and supports.

10. The method of claim one causing hyperemia and angiogenesis and cellular activation in the dermis of the skin containing regenerating the cellular network contained within including the blood vessels, hair follicles, sweat glands, collagen bundles, fibroblasts, nerves and lymph vessels and regenerates the subcutis which contains a network of collagen and fat cells as a result of improved facial outcome results from bolused subdermal therapies in the context of facials provided below the dermis, in the hypodermis or in bolused structures below the skin.
